(19) United States
(12) Patent Application Publication
(10) Pub. No.: US 2022/0361943 A1
(43) Pub. Date: Nov. 17, 2022

(54) SCREW-IN BIPOLAR ABLATION, MAPPING AND THERAPEUTIC CATHETER

(71) Applicant: University Health Network, Toronto (CA)

(72) Inventor: Kumaraswamy Nanthakumar, Mississauga (CA)

(21) Appl. No.: 17/744,483

(22) Filed: May 13, 2022

Related U.S. Application Data
(60) Provisional application No. 63/188,444, filed on May 13, 2021.

(51) Int. Cl. A61B 18/14; A61B 5/283; A61B 5/367; A61B 5/00

(52) U.S. Cl. CPC A61B 18/1492; A61B 5/283; A61B 5/367; A61B 5/6856; A61B 5/6857; A61B 2018/126

(57) ABSTRACT

Various embodiments are described herein for a bipolar catheter that generally comprises: a catheter body having a distal end portion and a proximal end portion; a first electrode at the distal end portion, the first electrode being on a spiral structure for rotational insertion into a physiological target region; a second electrode at the proximal end portion and spaced apart from the first electrode; and first and second electrode terminals spaced apart from one another at the proximal end portion and electrically coupled to the first and second electrodes respectively. The first and second electrodes are configured to function as active and dispersive electrodes respectively, or vice-versa. Also described are various embodiments of methods which generally include coupling the bipolar catheter to a signal generator; inserting the bipolar catheter at a physiological target region; and performing the procedure.

### CROSS-REFERENCE

This application claims the benefit of U.S. Provisional Patent Application No. 63/188,444 filed May 13, 2021; the entire contents of Patent Application 63/188,444 is hereby incorporated by reference.

### FIELD

Various embodiments are described herein that generally relate to an ablation catheter with an anode and a cathode built-in at the tip of the catheter allowing for bipolar ablation using a single catheter as well as transmural mapping and pacing.

### BACKGROUND

The following paragraphs are provided by way of background to the present disclosure. They are not, however, an admission that anything discussed therein is prior art or part of the knowledge of persons skilled in the art.

Radiofrequency Ablation technology is widely used to treat some of the most common and critical drug resistant arrhythmias. Currently catheters can be positioned either on the endocardium or more rarely on the epicardium and from this surface approach clinicians treat arrhythmias originating from within the myocardium. Unipolar ablation is the most commonly used ablation method in treating incessant arrhythmias. The main objective during ablation, is to obtain a uniform and sufficiently transmural lesion on the desired target of arrhythmia sources.

Multiple factors dictate the efficacy of the unipolar ablation such as: contact force, power delivery, duration, irrigation and system impedance. Furthermore, when the source of arrhythmia is intramural and far from the surface, unipolar ablation may fail to deliver enough energy to create a transmural lesion. For example, it is difficult for current unipolar ablation techniques to provide deep lesions and target intramural arrhythmia foci.

As a result, bipolar ablation is used to create a transmural lesion by typically holding catheters on opposing surfaces across the myocardium. Bipolar ablation leads to a lower system impedance resulting in deeper and wider transmural lesions. Although effective, the use of bipolar ablation is limited due to safety issues and the fact that 2 separate conventional catheters have to be held across the myocardium such that they are aligned and diametrically opposed to one another, which can be quite challenging. Achieving this is not always possible since any other location than the septum requires a catheter on the epicardial surface (an approach that is risky and not always possible) and constantly keeping the 2 catheters opposing each other throughout the cardiac motion.

### SUMMARY OF VARIOUS EMBODIMENTS

In accordance with one aspect of the teachings herein, there is provided a bipolar catheter comprising: a catheter body having a distal end portion and a proximal end portion; a first electrode at the distal end portion, the first electrode is on a portion of a spiral structure for rotational insertion into a physiological target region; a second electrode at the proximal end portion and spaced apart from the first electrode; and first and second electrode terminals spaced apart from one another at the proximal end portion and electrically coupled to the first and second electrodes respectively, wherein the first and second electrodes are configured to function as active and dispersive electrodes respectively, or the first and second electrodes are configured to function as dispersive and actives electrodes, respectively.

In at least one embodiment, the first electrode is part of a coil electrode and the active electrode has an insulated portion and a conductive surface portion.

In at least one embodiment, the dispersive electrode is a ring electrode that is disposed about a circumference of the distal end portion.

In at least one embodiment, the bipolar catheter comprises a conduit that has a first end that is connectable to a source of one or more medical agents and a second end at the distal end of the catheter and during use the one or more medical agents flow through the conduit and are delivered at the distal end portion of the bipolar catheter.

In at least one embodiment, the bipolar catheter comprises a conduit that has a first end that is connectable to a fluid source and a second end at the distal end of the catheter and during use fluid from the fluid source is flowed through the conduit and delivered at the distal end portion of the bipolar catheter for irrigation and/or cooling.

In at least one embodiment, the bipolar catheter comprises first and second conduits with the first conduit having a first end that is connectable to a source of one or more medical agents and a second end that is disposed at the distal end of the bipolar catheter and the second conduit having a first end that is connectable to a fluid source and a second end that is disposed at the distal end of the bipolar catheter and during use the one or more medical agents flow through the first conduit and are delivered at the distal end portion of the bipolar catheter and/or fluid from the fluid source is flowed through the second conduit and delivered at the distal end portion of the bipolar catheter for irrigation.

In at least one embodiment, the first electrode has pores and the first electrode is connected to the conduit for receiving the one or more medical agents during use.

In at least one embodiment, the second electrode has a porous structure and the second electrode is connected to the conduit for receiving the fluid for irrigation and/or cooling during use.

In at least one embodiment, the catheter further comprises an adjustment mechanism that is mechanically coupled to the first electrode and configured to controllably adjust a variable depth of penetration of a tip of the first electrode away from or closer to an end face of the catheter body at the distal end portion.

In at least one embodiment, the adjustment mechanism includes a micrometer that is configured to controllably adjust a variable depth of penetration of the tip of the first electrode during use.

In at least one embodiment, fluoroscopic markers are attached to the spiral structure to allow for visual confirmation of the depth of the tip of the spiral structure during use via fluoroscopic imaging.

In at least one embodiment, the bipolar catheter further comprises a temperature sensor at the distal end portion of the catheter body.

In at least one embodiment, the bipolar catheter further comprises a second pair of electrodes located at the distal end of the bipolar catheter and configured for measuring voltages thereat during use.

In at least one embodiment, the spiral structure further comprises a plurality of additional electrodes for providing at least two bipoles for performing high density intramural bipolar mapping or unipolar mapping.

In at least one embodiment, the physiological target region comprises cardiac tissue and the bipolar catheter is configured for use in bipolar ablation, transmural mapping, pace mapping, cardiac debulking and/or monitoring lesion formation.

In another aspect, in accordance with the teachings herein, there is provided at least one embodiment of a method of performing a procedure using a bipolar catheter, wherein the method comprises: coupling the bipolar catheter to a signal generator, the bipolar catheter being defined according to any one of the embodiments described herein; inserting the bipolar catheter at a physiological target region: and performing the procedure.

In at least one embodiment, the bipolar catheter is inserted at an initial target region until the second electrode contacts a surface of the physiological target region.

In at least one embodiment, the physiological target region comprises cardiac tissue and the procedure comprises bipolar ablation, transmural mapping, pace mapping, cardiac debulking and/or monitoring lesion formation.

In at least one embodiment, the bipolar catheter further comprises a temperature sensor at a distal end portion of the catheter body and the procedure comprises performing bipolar ablation or tissue debulking and the method comprises measuring a temperature at the target tissue region and stopping the bipolar ablation when the measured temperature is higher than a temperature threshold.

In at least one embodiment, the procedure comprises performing bipolar ablation or tissue debulking and the method comprises measuring an impedance at a distal end portion of the bipolar catheter and performing the bipolar ablation or tissue debulking while the measured impedance is within an effective operating range.

In at least one embodiment, the procedure comprises performing bipolar ablation or tissue debulking and the method comprises measuring impedance and comparing the measured impedance to an impedance threshold to determine when the ablation or debulking has been done for a sufficient amount of time.

In at least one embodiment, the procedure comprises performing bipolar ablation or tissue debulking and the method comprises measuring a first voltage before the procedure and a second voltage after the procedure, determining a magnitude reduction for the second voltage compared to the first voltage and determining that the medical procedure was successful when the magnitude reduction is greater than a magnitude reduction threshold.

In at least one embodiment, the procedure comprises performing bipolar ablation or tissue debulking and the method comprises measuring voltage during the procedure and determining that the procedure is complete when there is a loss in signal capture.

In at least one embodiment, the procedure comprises delivering fluid to the physiological target region to provide cooling and/or reduce formation of char during RF ablation.

In at least one embodiment, the procedure comprises delivering one or more medical agents to the physiological target region.

In at least one embodiment, the one or more medical agents comprise one or more therapeutic agents, one or more diagnostic agents and/or one or more marker agents.

In at least one embodiment, the one or more medical agents comprise pharmaceuticals, antiarrhythmics, MRNA and DNA to reprogram cells in the physiological target region, stem cells, viral vectors to reprogram existing cells in the physiological target region, biological pacemakers, radiology markers for later targeting therapy with radiation or a gamma knife and/or biological reporters.

In another aspect, in accordance with the teachings herein, there is provided at least one embodiment of a use of a bipolar catheter, wherein the bipolar catheter is defined according to any one of the embodiments described herein, and is configured to perform a procedure comprising bipolar ablation, transmural mapping, pace mapping, cardiac debulking and/or monitoring lesion formation.

Other features and advantages of the present application will become apparent from the following detailed description taken together with the accompanying drawings. It should be understood, however, that the detailed description and the specific examples, while indicating preferred embodiments of the application, are given by way of illustration only, since changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A is an illustration of an example embodiment of a bipolar catheter containing an electrode on a spiral structure in accordance with the teachings herein.

FIG. 1B is an illustration of an example use of the bipolar catheter of FIG. 1A.

FIG. 2A is an illustration of an alternative example embodiment of a bipolar catheter containing an electrode on a spiral structure in accordance with the teachings herein.

FIG. 2B is an illustration of an alternative example embodiment of a helical electrode that can be used with a bipolar catheter described herein.

FIG. 2C is an illustration of an example use of the bipolar catheter of FIG. 2A.

FIGS. 3A-3C are illustrations of another alternative example embodiment of a bipolar catheter containing an electrode on a spiral structure in accordance with the teachings herein.

FIG. 4 is a block diagram of an example embodiment of a system for use with at least one embodiment of a bipolar catheter described in accordance with the teachings herein.

FIG. 5 is a flowchart of an example embodiment of a method of performing a cardiac procedure using an embodiment of the bipolar catheter described herein.

FIG. 6A is a vertical cross-sectional illustration of the bipolar catheter of FIG. 1A at a beginning stage of performing bipolar catheter ablation on a lesion.

FIG. 6B is a vertical cross-sectional illustration of the bipolar catheter of FIG. 1A at the end of performing bipolar catheter ablation on the lesion of FIG. 6A.

FIG. 6C is a bottom cross-sectional illustration of the bipolar catheter of FIG. 1A at the end of performing bipolar catheter ablation on the lesion of FIG. 6A.

FIGS. 7A-7C are illustrations of bipolar voltage reduction, signal capture and measured impedance while performing RF ablation using one of the bipolar catheters described in accordance with the teachings herein.

FIGS. 8A-8B are illustrations showing an alternative example embodiment of a bipolar catheter having a different arrangement of electrodes on a spiral structure in accordance with teachings herein.

FIG. 9 is an illustration showing an example use of the bipolar catheter of FIG. 3 for cardiac debulking.

FIG. 10 is an illustration showing an example use of the bipolar catheter of FIG. 3 for determining a suitable location for electrode placement for a pacemaker.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical signal, electrical connection, or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

In addition, it should be noted that the phrases “at least one of X and Y” or “X, Y or a combination thereof” is intended to mean X, Y or X and Y.

It should also be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term that it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

To overcome the limited amount of energy penetrating into the myocardium (during unipolar ablation) and to limit the safety issues posed when bipolar ablation is performed using two separate unipolar ablation catheters, there is provided, in accordance with a first aspect of the teachings herein, a bipolar catheter having two electrodes in which the distal-most electrode is on a spiral structure, which, can be screwed into tissue thereby enabling increased energy delivery to tissue for ablation. The electrode on the spiral structure may be a coil electrode or a screw electrode. The bipolar catheter may also be referred to as a bipolar ablation catheter and more particularly a screw-in bipolar ablation catheter. By inserting the electrode along with the spiral structure into the heart tissue, the delivered energy will disperse from within the myocardium. The screw-in bipolar catheter can be connected to an energy source, such as an RF generator, for example, that is commonly available to medical practitioners who perform RF ablation. Accordingly, the screw-in bipolar catheter can be used to perform deep and uniform lesions using RadioFrequency (RF) ablation.

Advantageously, the bipolar catheter allows for delivery of bipolar ablation from a single catheter without having to access 2 different chambers or opposing myocardial surfaces. This is in contrast to traditional methods that use conventional unipolar ablation catheters to deliver energy to the heart tissue surface where the energy will have to penetrate through the heart tissue, and thus the localization and amount of ablation will suffer.

Furthermore, the use of a spiral structure at the distal end of the bipolar catheter, in accordance with the teachings herein, allows for stability when the distal end of the bipolar catheter is inserted into tissue, and hence the location of the electrodes of the bipolar catheter can be maintained desired positions. The screw-tip may have a preset length which is selected so that the distal end of the bipolar catheter is sufficiently anchored in tissue so that it does not move during use even in the presence of cardiac muscle or tissue movement. An ablation catheter where an electrode is located on a spiral structure that is inserted into the myocardium will also increase the probability that a lesion is formed from within the myocardium during the ablation process, without damaging any vulnerable structures such as valves, for example, and also allow for the size and depth of the lesion to be more accurately controlled.

In accordance with a second aspect of the teachings herein, in at least one embodiment, the bipolar catheter can further include a control mechanism that can be used to control depth titration when the tip of the spiral structure is inserted into a portion of the heart. For example, the control mechanism can be a micrometer-based control mechanism that can be used to fine-tune the length of the penetration depth of the screw-tip in cardiac tissue. This allows for fine placement of the tip of the electrode on the spiral structure so that the bipolar catheter can be used to more accurately ablate a desired cardiac region. For example, by increasing the penetration depth of the electrode on the spiral structure, a deeper lesion may be formed while optimizing the amount of RF energy that is delivered to the patient and localizing the ablation to the targeted area.

In accordance with a third aspect of the teachings herein, in at least one embodiment, the bipolar catheter may also include a perforated structure for at least one of the electrodes at the distal end that are used to provide RF ablation. In such embodiments, the perforated structure may be used for irrigation during cardiac treatment, such as RF ablation, to cool down the region of the heart that is receiving the cardiac treatment and also reduce the formation of any char. In at least one of these embodiments, one of the electrodes has the perforated structure.

In accordance with a fourth aspect of the teachings herein, in at least one embodiment, the bipolar catheter may also include a delivery conduit for delivering an object, such as medication, into an organ such as the heart. After the spiral structure is used to anchor the bipolar catheter at a desired location on or within the organ, the delivery conduit may be used to provide chemicals, pharmaceuticals and/or cells (e.g., stem cells) to the desired location of the organ.

It should be noted that embodiments of the bipolar catheter may be formed by combining the embodiments having the first aspect with embodiments having any of the second to fourth aspects described above.

In accordance with another aspect of the teachings herein, in at least one embodiment, the bipolar catheter may be used for transmural mapping. Accordingly, the bipolar catheter may also be referred to as a bipolar mapping catheter. For example, the variable depth penetrating bipolar catheter may be used to perform transmural mapping by employing the electrode on the spiral structure, which can be used in a bipolar mapping mode. This may also be beneficial for mapping the intramural space since conventional unipolar catheters can only be used to map the endocardium and epicardial surfaces, and cannot map the intramural surface.

In accordance with another aspect of the teachings herein, in at least one embodiment, the bipolar catheter may be used for pace mapping. Accordingly, the bipolar catheter may also be referred to as a bipolar pace mapping catheter. For example, the bipolar catheter can be used to provide stimulus signals for pacing rather than RF signals for ablation. The stimulus signals may have certain characteristics, as is known by those skilled in the art, to be able to identify one or more sources of different types of abnormal heart rhythms such as, but not limited to, tachycardia, brachycardia, and atrial or ventricular fibrillation, for example. Heretofore, no one has previously been able to perform pacing from the intramural space as no one had previously been able to access this space before. However, with the bipolar catheters described herein, the intramural space can now be accessed.

In accordance with another aspect of the teachings herein, in at least one embodiment, the bipolar catheter may also be used for tissue debulking by inserting the bipolar catheter at a desired location and applying RF energy to remove a desired amount of tissue.

In accordance with another aspect of the teachings herein, in at least one embodiment, the bipolar catheter may be used for various other applications such as tissue debulking, monitoring lesion progress and/or determining an appropriate location for applying pacemaker leads.

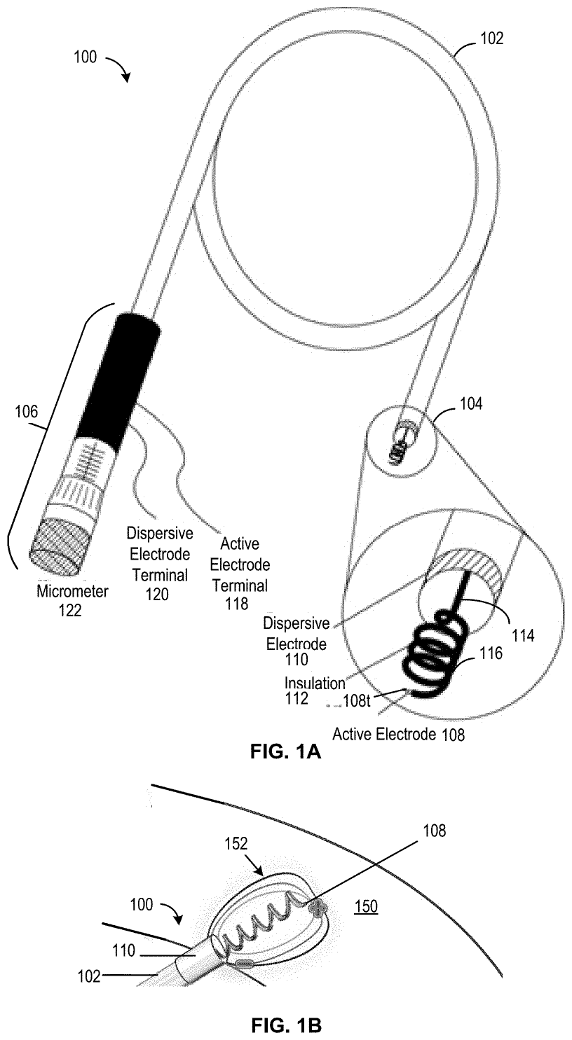

Referring now to FIG. 1A, shown therein is an illustration of a bipolar catheter 100 containing an electrode with on a spiral structure, in accordance with the teachings herein, for removable rotational insertion into tissue, such as cardiac tissue, for example. The bipolar catheter 100 comprises a catheter body 102 having a distal end portion 104, and a proximal end portion 106. At the distal end portion 104 the bipolar catheter 100 comprises a first electrode which can be referred to as an active electrode 108, and a second electrode which can be referred to as a dispersive electrode 100. The active electrode 108 is on the spiral structure for removable rotational insertion into tissue. The proximal end portion 106 includes active and dispersive electrical terminals 118 and 120 that are spaced apart from one another and electrically coupled to the active and dispersive electrodes 108 and 110.

The bipolar catheter 100 may include a first conductor that is connected to the active electrode 108 and the active electrode terminal 118 and a second conductor that is connected to the dispersive electrode 110 and the dispersive electrical terminal 120. The active and dispersive electrode terminals 118 and 120 may be electrically connected to a signal generator, such as an RF generator, for example.

Referring now to FIG. 1B, shown therein is an illustration of an example use of the bipolar catheter 100 of FIG. 1A in which it is performing RF ablation in cardiac tissue 150. The active terminals 118 and 120 (not shown) are connected to signal generator, such as an RF source, and an electric field 152 is generated due to the potential difference across the active and dispersive electrodes 108 and 110.

Referring again to FIG. 1A, the active electrode 108 is formed using a conductive wire that has a straight portion that extends out of an opening in the catheter body 102 at the distal end portion 104 and transitions into a helical portion 116. The active electrode 108 may be referred to as a screw-profile electrode. The conductive wire 114 includes an insulation portion that is covered in an insulation layer 112 and a conductive portion that is disposed at the tip of the conductive wire 114, in this example embodiment. The conductive portion is the portion of the conductive wire 114 that is not insulated to RF energy to tissue during use.

In at least one alternative embodiment, the electrode 108 may be used as a reference electrode with the other electrode 110 acting as the active electrode with both of the electrodes 108 and 110 having separate connections to the electrical terminals 118 and 120 and the electrical terminals 118 and 120 are coupled to the RF such that their polarities are reversed compared to what is shown in FIG. 1B, for example.

As the exposed end of the active electrode 108 may potentially cause a short-circuit when it may be closer to the dispersive electrode 110 (i.e., return electrode) than intended, the segment of the active electrode 108 that is closer to the dispersive electrode 110 was coated and covered with a high impedance material. For example, the high impedance material may be liquid electrical tape or a rubber spray coating.

In this example embodiment, the spiral structure is provided by a coil electrode (which may also be referred to as a screw electrode) where a majority of the electrode may be insulated except for certain regions such as the tip. Alternatively, in at least one embodiment, the spiral structure is provided by a screw with threads that extend a sufficient extent in the radial direction from the main body of the screw to allow for stable placement in tissue. For the active electrode 108 to latch into tissue, such as cardiac tissue, the spiral structure may have a helical shape. The helical shape allows for controlled depth titration.

In at least one embodiment, the spiral structure of the bipolar catheter 100 may be implemented so that it is possible to retract the spiral structure without causing tissue damage (e.g., cell necrosis). However, in at least one alternative embodiment, the radial extent of the spiral structure may be chosen to be a bit larger and/or stiffer material may be chosen to make the spiral structure so that small pieces of cardiac tissue/muscle may be obtained when the spiral structure is removed from the cardiac tissue/muscle thereby allowing a cardiac biopsy to be performed. For example, in such embodiments for performing cardiac biopsies, material such as mp35n may be used which is high tensile strength cobalt nickel alloy and the spiral structure is about 4 French, e.g., it has around 5-7 mm² of surface area, and the ring electrode may have a surface area of 4-5× that of the spiral structure. However, it should be noted that these same materials and dimensions can be used for bipolar catheters which are used in applications other than cardiac biopsies.

In at least one embodiment, the ability of the bipolar catheter to be used as a biopsy tool may also be combined with its ability to be used as a mapping tool so that: (1) a desired location for a desired biopsy can be correctly found using the bipolar catheter 100 during mapping, (2) after the proper location is determined, the spiral structure of the bipolar catheter can be rotatably extended into the desired location and (3) the spiral structure of the bipolar catheter can then be pulled back (without rotation) to obtain a cardiac biopsy.

In at least one embodiment, the length of the portion of the spiral structure having the active electrode 108 may be about 10 mm.

In at least one example embodiment, using a 0.032″ (0.8128 mm) outer diameter of a titanium wire, a helical coil electrode was manually made by coiling the wire against a 19 gauge syringe needle. To maintain uniformity of the helical coil, the tip of the syringe needle was cut off and the remaining part of the needle was used. After this process, a coiled region of 10 mm was formed. To increase the toughness of the coil electrode, it was exposed to a high heat source for 5-10 minutes and rapidly cooled. This may be done by exposing the coil electrode to an open flame that may be sustained through a Bunsen burner using propane gas.

The dispersive electrode 110 is spaced apart from the active electrode 108. In this example embodiment, the dispersive electrode 110 is a ring electrode that is positioned along the outer circumference near the end of the catheter 102 and is spaced apart from the active electrode 108. The dispersive electrode 110 may act as a reference electrode depending on the usage of the bipolar catheter 100.

The shape and size of the dispersive electrode 110 can influence the forming of a lesion when the bipolar catheter 100 is used for ablation. For example, the size of the dispersive electrode 110 may be selected to minimize the outer diameter size of the catheter body 102 while still maintaining a reasonable amount of impedance for increased energy delivery during RF ablation.

In addition, the shape and size of the dispersive electrode 110 has a role in making sure that the RF generator unit (e.g., RF source) that is used during ablation does not fail on impedance (i.e., the impedance at the distal end portion of the bipolar catheter 100 remains in an effective operating range). This is because current RF generators will cut out when impedance rises above 200 ohms as safety feature.

Another consideration may be the distance (e.g., spacing) between the distal end of the dispersive electrode 110 and the proximal end of the electrode tip 108t may be selected to be smaller to record smaller potentials, such as about 40 to 75 microvolts, for example, if any recorded noise be can reduced below those levels.

In at least one embodiment, the outer diameter size of the ring electrode may range from about 5 mm and 7 mm.

In at least one embodiment, the length of the ring electrode may range from about 4 mm to about 25 mm.

In at least one embodiment, the ring electrode may have a surface area that is about 4 to 6 times the exposed (i.e., conductive) surface area of the spiral structure.

The catheter body 102 may be implemented using a cable that is generally made of medical grade material.

Furthermore, in various embodiments of the bipolar catheter discussed throughout this description, the catheter body 102 may include a guide wire, such as a 0.035 guide wire, so that the bipolar catheter can travel “over the guide wire” so that the distal end of these bipolar catheter can be directed to a physiological target region.

In this example embodiment, the proximal end portion 106 of the bipolar catheter 100 also includes an adjustment mechanism that is mechanically coupled to the active electrode 108 and configured to extend a tip 108t of the active electrode 108 further away from or closer to an end face of the catheter body 102 at the distal end portion 104. For example, the proximal end portion 106 can include a knob that the user turns to linearly and rotationally move the active electrode 108 (e.g., the spiral structure can rotate as it is extended or retracted).

In at least one embodiment, the adjustment mechanism includes a micrometer screw gauge 122 that is configured to controllably adjust a variable depth of penetration of the tip 108t of the active electrode 108.

In at least one embodiment, fluoroscopic markers can be added to the spiral structure so that it is possible to determine the depth of insertion of the spiral structure. For example, fluoroscopic markers can be added to the tip of the spiral structure to allow for visual confirmation of the depth of the tip of the spiral structure during use by based on how far the fluoroscopic markers are the dispersive electrode by looking at fluoroscopic images.

In at least one embodiment, the bipolar catheter 100 further includes a temperature sensor (not shown) that is disposed at the distal end portion 104 of the catheter body 102. The temperature sensor is used to obtain temperature measurements during use of the bipolar catheter 100 to make sure that an appropriate amount of heat to the tissue is being applied during RF ablation. Also, if the bipolar catheter is being used for bipolar ablation or tissue debulking at a target tissue region, the temperature at the target tissue region can be measured and the bipolar ablation or tissue debulking can be stopped when the measured temperature is higher than a temperature threshold.

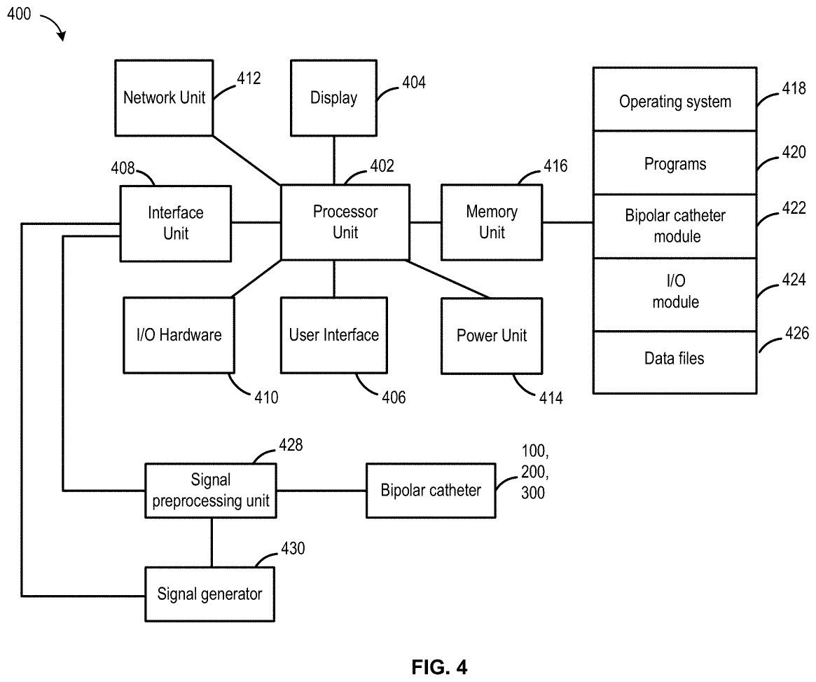

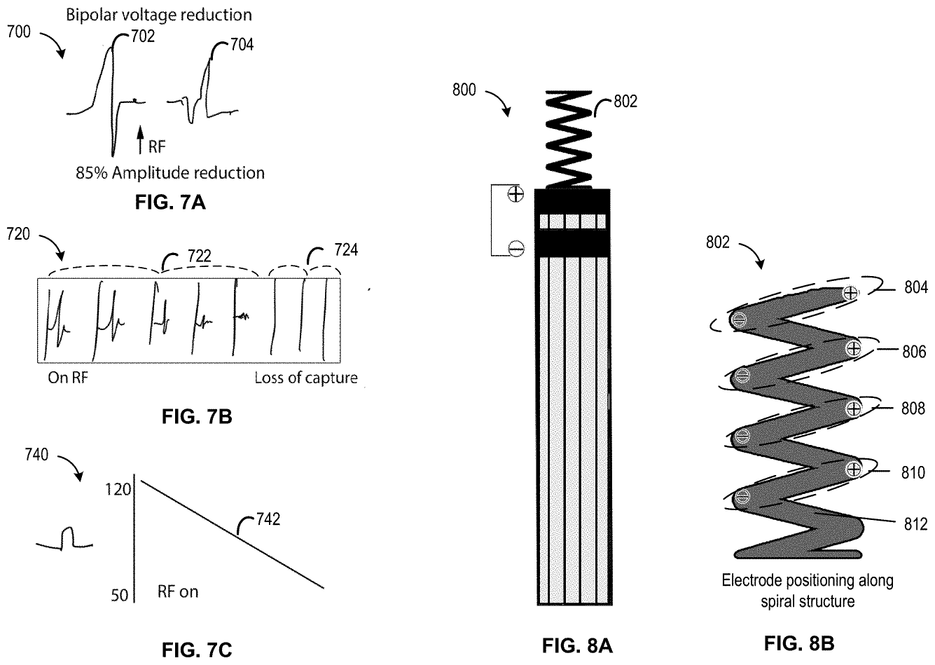

In at least one embodiment, the bipolar catheter 100 has an intramural bipolar voltage and impedance measurement capacity. In such embodiments, the bipolar catheter 100 may be used to monitor progression of RF ablation as is explained in further detail with respect to FIGS. 7A-7C. In such embodiments, the bipolar catheter 100 may be used with a bipolar catheter system 400 an example of which is described with respect to FIG. 4.

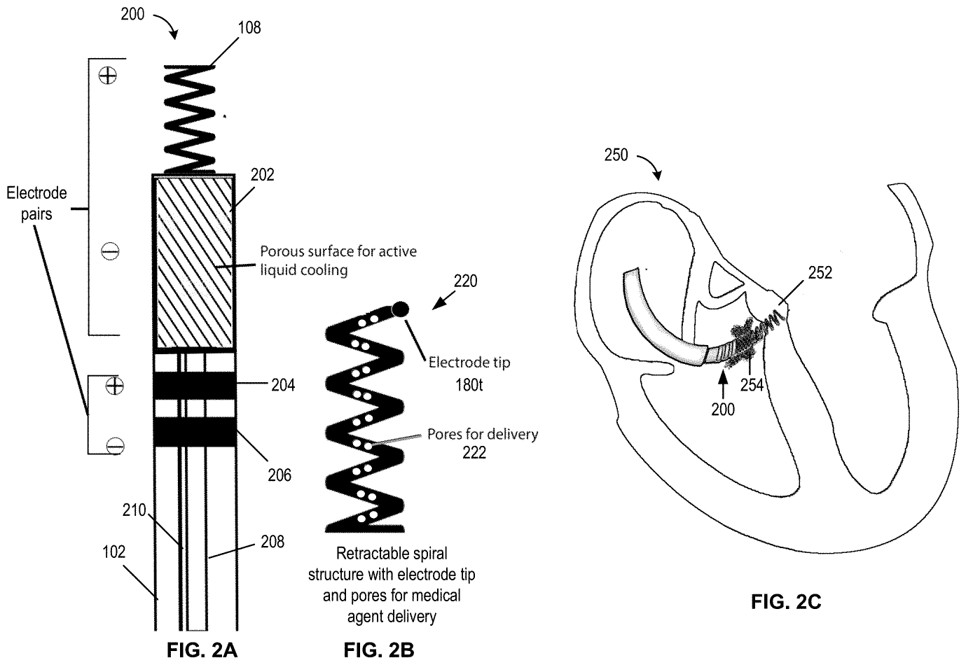

Referring now to FIG. 2A, shown therein is an illustration of an alternative example embodiment of a bipolar catheter 200 containing an electrode on a spiral structure in accordance with the teachings herein. The bipolar catheter 200 is similar to the bipolar catheter 100 and includes all of the same components and can have alternative embodiments as was described for the alternative embodiment of the bipolar catheter 100 except that the bipolar catheter 200 includes an alternative structure for one of the electrodes. In this case, the electrode 202, which may act as the dispersive electrode depending on the polarity of the voltage difference across electrodes 108 and 202, is made using a porous structure with many little apertures or slits (both not shown) which serves two purposes. Furthermore, the bipolar catheter 200 includes a conduit 208 that is disposed within the catheter 200 in a longitudinal fashion for allowing for fluid to be delivered to the distal end of the catheter 200. The fluid that can be delivered may be an irrigation fluid, a cooling fluid or a fluid that provides for both irrigation and cooling.

Firstly, the porous structure allows for the conductive surface area of the electrode 202 to be increased compared to having a smooth surface which is advantageous as this provides a greater surface area to receive the return Electric fields that are generated when there is a potential difference (e.g., voltage difference) across the electrodes 108 and 202. This results in the Electric field being more tightly concentrated around the electrode 108 so that they do not spread out as much from the electrodes 108 and 202 (see FIG. 1B) and this allows any RF ablation that is being done to be more focused and concentrated on a particular region of interest and reduce any potential damage to any nearby structures. In addition, in an alternative embodiment, electroporation may be used as the energy source, rather than RF energy, as electroporation helps to protect injury to blood vessels.

Secondly, the porous structure of the electrode 202 allows for irrigation. Accordingly, in embodiments of the bipolar catheter that employ a porous structure for the electrode (e.g., the electrode is perforated) that is disposed closer to the surface of the catheter body 102, there is included the conduit 208 within the catheter body 102 that is fluidically coupled to the porous electrode 202 to send a fluid therethrough which then irrigates the region proximal to the electrode 202. An example of this is shown in FIG. 2C where the bipolar catheter 200 has been inserted into region 252 of heart 250 where fluid 254 is dispersed during RF ablation. The fluid that is dispersed from the electrode 202 can be used to reduce any increase in temperature that might result due to RF ablation. The fluid that is dispersed from the electrode 202 can also be used to prevent or reduce the formation of any char that might be crated during the RF ablation process. Therefore, with the bipolar catheter 200, more RF power may be delivered to a physiological target region during RF ablation since irrigation can be used to prevent excessive temperature increase and the formation of char. The fluid that may be used includes, but is not limited to, Normal Saline (NS) to prevent coagulation formation, for example. For instance, ½ NS may be used which increases impedance and also re-routes more of the power to the electrode on the spiral structure. The physiological target region may include muscle, a clot, a tumour and/or tissue. The physiological target region may also be referred to as a target region herein.

In such embodiments, the proximal end of the bipolar catheter 200 is coupled to a fluid source (not shown) which is in turn coupled to the fluid conduit 208 within the catheter body 102. There may also be a fluid control valve (not shown) at the proximal end (e.g., end portion 106) of the bipolar catheter 200 to allow for controlling the flow of the fluid from the fluid source through the fluid conduit 208 to the porous electrode 202 for dispersion of the fluid in the region that is being ablated.

In this example embodiment, there are also an additional pair of electrodes 204 and 206 so that the bipolar electrode 200 has two pairs of bipolar electrodes including a first pair of bipolar electrodes 108 and 202 and a second pair of bipolar electrodes 204 and 206. These second pair of bipolar electrodes 204 and 206 may be used for recording electrophysiological signals from an organ such as, but not limited, the heart, for example. It should be noted that the electrodes 204 and 206 are optional and may not be used in other embodiments of bipolar catheters which use at least one porous electrode.

The bipolar catheter 200 also includes electrode wires 210 that are coupled to the electrodes 108 and 202 as well as electrodes 204 and 206 (when they are included in the bipolar catheter 200). The electrode wires 210 can be located such that they are adjacent to an exterior portion of the conduit 208.

It should be noted that in an at least one alternative embodiment, the bipolar catheter 200 may deliver coolant to the target region for providing cryo-therapy to kill tissue in the target region. This may be done using the fluid conduit 208. The coolant may be used as an alternative to using RF power to ablate tissue in the target region.

Referring now to FIG. 2B, shown therein is an illustration of an alternative example embodiment of a coil (i.e., helical) electrode 220 that can be used with a bipolar catheter described herein. The coil electrode 220 includes at least one pore, aperture or hole 222 for allowing a fluid to be dispersed therefrom. The coil electrode 220 may be used in a few different ways. Firstly, the helical electrode 220 may be used with the bipolar catheter 200 such that the fluid conduit 208 is also coupled to the pores 222 of the coil electrode 220 so that fluid for irrigation, as described for the porous electrode 202, may also be dispersed using the pores 222 of the coil electrode 222. Alternatively, in at least one other embodiment, the coil electrode 220 may also be used with the bipolar catheter 200 or the bipolar catheter 100, or alternatives thereof, and there is a separate conduit that is coupled to the pores 222 of the coil electrode 220 so that pharmaceuticals (drugs), cells or other medical agents may be sent to the coil electrode 220 and dispersed therefrom to a target region, such as the myocardium, for example.

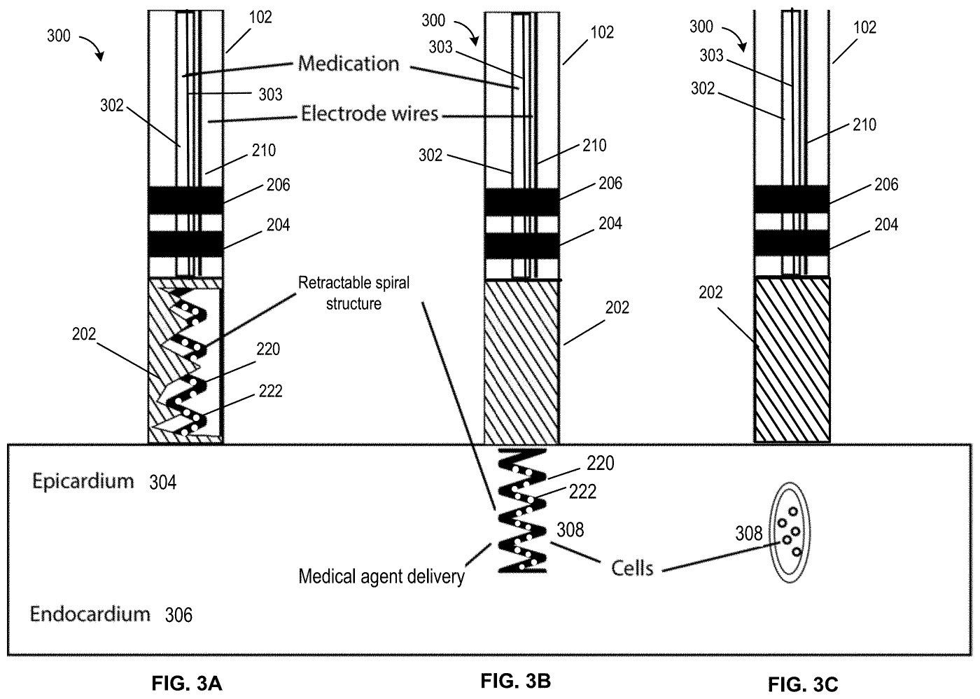

Referring now to FIGS. 3A-3C, shown therein are illustrations of another alternative example embodiment of a bipolar catheter 300 containing an electrode on a spiral structure in accordance with the teachings herein. The bipolar catheter 300 is similar to the bipolar catheter 200 and generally includes all of the components of the bipolar catheter 200 but it also includes a bifurcated conduit 302 to provide first and second conduits for delivery of irrigating fluids and/or medical agents, respectively, to a desired target region. Medical agents are defined as encompassing one or more therapeutic agents, one or more diagnostic agents and one or more marker agents. For example, medical agents include, but are not limited to, pharmaceuticals, antiarrhythmics, MRNA and DNA to reprogram cells in the target region, stem cells, viral vectors to reprogram existing cells in the target region, biological pacemakers, radiology markers for later targeting therapy with radiation or a gamma knife and/or biological reporters. Just as with the bipolar catheter 200, the electrodes 204 and 206 are optional. Also, in at least one alternative embodiment, the bipolar catheter 300 may include a solid ring electrode such as electrode 110 instead of the porous electrode 202.

The dual conduit 302 of the bipolar catheter 300 may run along the longitudinal axis of the catheter body 102 and has a central dividing wall 303 so that there are two conduits or lumens such that a first conduit is coupled to the porous electrode 202 for delivering a fluid, such as irrigation fluid thereto and a second conduit is coupled to an interior of the spiral structure for delivering one or more medical agents to the retractable screw electrode 220 which may then be ejected via the pores 222 into the surrounding region. This then allows for various combinations of functions to be performed. For example, the first conduit for providing irrigation fluid may be used so that irrigation is performed during RF ablation. Alternatively, only the second conduit may be used to provide one or more medical agents to the target region. In another alternative, the conduit for providing irrigation fluid may be used first so that irrigation is performed during RF ablation and then after the completion of RF ablation, the second conduit may be used to provide one or more medical agents to the target region. Alternatively, in other cases irrigation may be performed for another reason such as during clot buster infusion (e.g., TPA) for one or more congealed clots in one or more blood vessels.

In at least one alternative example embodiment, a single conduit may be used for the bipolar catheter 300 and coupled to the interior of the electrode 220. In this case the bipolar catheter may be coupled to just a medical agent source from which one or more types of medical agents flow to the electrode 220, via the conduit 302, and exit at the pores 222 of the electrode 220.

Alternatively, in at least one embodiment where the electrode 108 is used, an exit opening of the conduit 302 may be disposed adjacent to a first end of the spiral structure that is opposite a second end of the spiral structure where the second end includes the tip 108t of the electrode 108. One or more types of medical agents may then be flowed through the conduit to the exit opening for delivery into the target region.

In at least one embodiment, the longitudinal axis of the conduit 302, when having one or two conduits, may be aligned with the longitudinal axis of the spiral structure of the bipolar catheter or in other cases these axes may be misaligned.

The proximal end of the bipolar catheter 300 is coupled to an irrigation fluid source and a medical agent source (both not shown) which are in turn coupled to the first and second conduits, respectively, within the catheter body 102. There may also be an irrigation control valve and a medical agent control valve (both not shown) at the proximal end (e.g., end portion 106) of the bipolar catheter 300 to allow for controlling the flow of the irrigation fluid and the medical agents from the irrigation fluid source and the medical agent source, respectively, through the first and second conduits for dispersion/delivery at the distal end of the bipolar catheter 300.

Referring now to FIG. 3A, during use, the distal end of the bipolar catheter 300 that includes the electrodes 202 and 220 may be fed through certain access points such that the electrode 202 is adjacent to a cardiac structure such as the epicardium 304. Next, the spiral structure of the bipolar catheter can be rotatably extended, as was described for the bipolar catheter 100, into the epicardium 306 such that the tip of the electrode 220 moves closer to the endocardium 306 as is depicted in FIG. 3B. This allows the positioning of the distal end of the bipolar catheter 300 to be precisely located and anchored at a desired target region. As described previously, the desired target region may be located by performing mapping using the bipolar catheter 300. After the spiral structure of the bipolar catheter is deployed and inserted into the cardiac tissue (or muscle depending on the situation), medical agents 308, such as one or more types of cells (as explained earlier), in the second conduit 302 can be expelled or delivered to the desired target region. The spiral structure may then be rotatably retracted leaving behind the medical agents 308.

Referring now to FIG. 4, shown therein is a block diagram of an example embodiment of a catheter system 400 for use with at least one embodiment of a bipolar catheter described in accordance with the teachings herein, such as bipolar catheters 100, 200 or 300. The system 400 generally includes a processor unit 402, a display 404, a user interface 406, an interface unit 408, input/output (I/O) hardware 410, a network unit 412, a power unit 414, and a memory unit (also referred to as a “data store”) 416. The system 400 further includes a signal preprocessing unit 428 and a signal generator 430. In other embodiments, the system 400 may include additional components, fewer components or different components as long as the functionality described herein is provided.

In general, a user may interact with the system 400 to use one of the bipolar catheters described herein, e.g., bipolar catheter 100, 200 or 300, to perform a variety of functions including, but not limited to, RF ablation, mapping, pacing, debulking, biopsies and/or delivery of objects to a desired physiological target region. Accordingly, the majority of the components of the system 400 may be an electronic device or computer system that is coupled to the signal preprocessing unit 428, the signal generator and a particular bipolar catheter (as described herein) and used by a user, such as a medical practitioner or surgeon, to perform a medical procedure.

The processor unit 402 controls the operation of the system 400 and can be any suitable processor, controller, or digital signal processor that can provide sufficient processing power depending on the configuration, purposes, and requirements of the system 400. For example, the processor unit 402 may include a standard processor, such as the Intel Xeon processor. Alternatively, there may be a plurality of processors that are used by the processor unit 402, and these processors may function in parallel and perform certain functions. Therefore, the processor unit 402 is considered as having at least one processor.

The user interface 406 may be used to generate a set of windows or graphical user interface (GUI) screens that can be used to display certain information to a user and receive input parameters from the user. Alternatively, the user interface 406 may include input devices that a user can use to provide data inputs or control inputs to the system 400. These input devices include a keyboard, a mouse, a touchscreen, and the like. The user interface 406 can also include devices to provide an output to the user, such as the display 404 or a printer. The display 404 may be, but is not limited to, a computer monitor, an LCD display, or a touchscreen monitor.

The interface unit 406 can be any interface that allows the system 400 to receive data from or send control signals to other devices or hardware such as the signal preprocessing unit 428 and the signal generator 430. In some cases, the interface unit 408 can include at least one of a serial port, a parallel port, a USB port that provides USB connectivity or another suitable port or connections for sending and receiving signals.

The network unit 412 may be a standard network adapter such as an Ethernet or 802.11x adapter or another type of adapter. Accordingly, the network unit 412 can also include at least one of an Internet connection, a Local Area Network (LAN) connection, an Ethernet connection, a FireWire connection, a modem connection, or a digital subscriber line connection. Alternatively, or in addition, the network unit 412 include a wireless unit. For example, the network unit 412 can include a radio that communicates utilizing CDMA, GSM, GPRS, or Bluetooth protocol according to standards such as those in the IEEE 802.11 family (e.g., 802.11ac). The network unit 412 can be used by the operator unit 402 to communicate with other devices or computers.

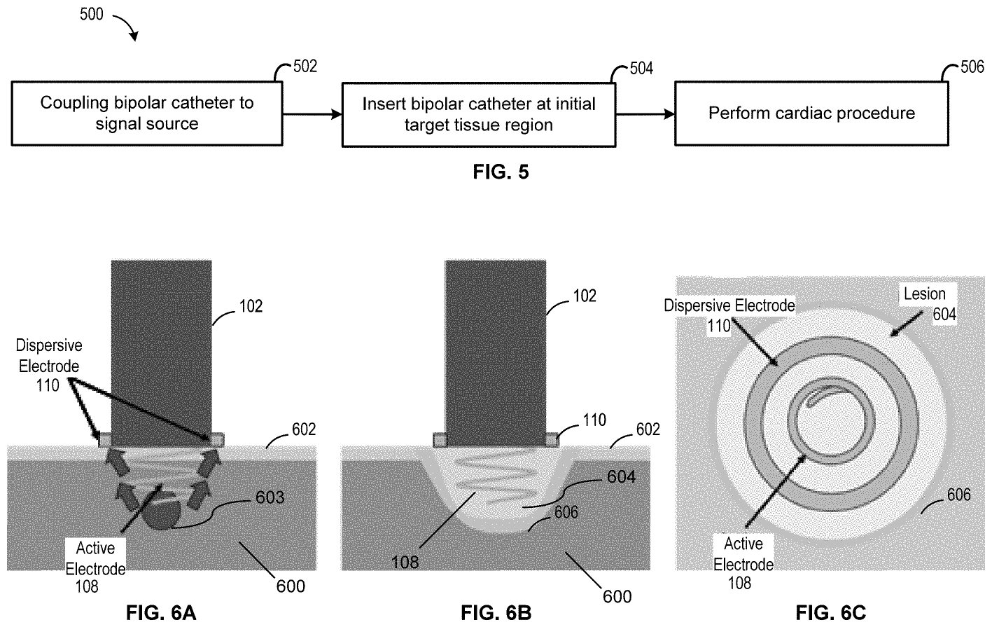

The memory unit 416 may store the program instructions for an operating system 418, programs 420, a bipolar catheter module 422, an I/O module 424 and data files 426. The programs 420 comprise program code that, when executed, configures the processor unit 402 to operate in a particular manner to implement various functions and tools for the pacemaker lead device 100. The bipolar catheter module 422 comprises program code that may be used to operate the bipolar catheter 100, 200, 300 in a certain mode to perform certain functions as is described in more detail with respect to FIGS. 5 to 10. The I/O module 424 includes programs for receiving input data from the user or any data obtained by the bipolar catheters 100, 200, 300 that are preprocessed by the signal preprocessing unit 428. The I/O module 424 may also be used to provide output data to the user, which may be done through outputting text and/or graphics to the display 404 and/or user interface 406 or by sending electronic messages and data via the network unit 412. Any data that is obtained by the bipolar catheter 100, 200, 300 may be stored in the data files 426. Other items such as operating parameters and/or calibration parameters may be stored in the data files 426.

The signal preprocessing unit 428 may be used to preprocess data that is sensed using the bipolar catheter 100, 200, or 300 from a patient or subject, which may be done in response to certain stimuli. Accordingly, the signal preprocessing unit 428 may also be used to control the timing for data acquisition. Accordingly, the signal preprocessing unit 428 comprises hardware circuitry that is used to record data sensed by the bipolar catheter 100, 200, or 300 from the patient or subject. For example, the signal preprocessing unit 428 may contain at least one amplifier, at least one filter, and an Analog Digital Converter (which may have multiple channels) for amplifying, filtering, and digitizing the sensed cardiac signals. Conventional amplification and filtering may be used.

The signal preprocessing unit 428 also includes circuitry for preprocessing any stimulus signals and RF signals that are generated by the signal generator 430. The signals generated by the signal generator 430 may require amplification and filtering and also conversion from digital to analog before being sent to the active and reference electrodes of the bipolar catheter 100, 200, 300. Accordingly, the signal preprocessing unit 428 may include separate or programmable hardware that is used for preprocessing signals received from the signal generator 430 where the hardware may include one or more amplifiers, one or more filters and digital to analog convertors (DACs) or a multi-channel DAC.

The signal preprocessing unit 430 may also include isolation circuitry and some form of circuit breaker of cutoff switches to prevent sending stimulus signals that are too powerful due to glitches, voltage spikes or current spikes to the patient/subject which may injure them as well as protecting other circuitry and hardware from such conditions.

The signal generator 430 may be used to provide stimulus signals for pacing and/or mapping to the bipolar catheter 100, 200, 300 that is being used in conjunction with the system 400. The signal generator 430 may also be used to generate RF signals. For example, the signal generator 430 may generate RF signals that are sent to the bipolar catheter 100, 200, 300 which is then used for ablation or debulking.

In at least one embodiment, electroporation may be used by the signal generator 430. When electroporation is used DC current with specific waveforms may be used instead of RF energy.

Referring now to FIG. 5, shown therein is an example embodiment of a method 500 of performing a cardiac procedure using any embodiment of the bipolar catheter described herein. For example, the bipolar catheter may be used for performing RF ablation, transmural mapping, pace mapping, debulking, monitoring progress of RF ablation and/or aiding in placement of pacemaker electrodes. One of the bipolar catheters described herein may be used for performing the method 500 (depending on the medical procedure being performed) as well as the system 400.

At 502, the method 500 comprises coupling the bipolar catheter to a signal source. This may also include selecting the signal parameters in cases where the signal parameters are not preset. This may be done through a user interface as was described for system 400. When performing RF ablation, the signal source may be operated as an RF generator. When performing transmural mapping or pace mapping, the signal source may be operated as a signal generator that is capable of generating signals used in performing transmural or pace mapping.

At 504, the method 500 comprises inserting the bipolar catheter at an initial target cardiac tissue region, which may generally involve rotatably inserting the spiral structure of the bipolar catheter, until the other electrode touches the tissue surface or is just above the tissue surface (for example within about 2 to 4 mm of the surface when performing ablation, mapping or pacing). In this case, the cardiac tissue region should be understood as including cardiac muscle.

At 506, the signal source, such as signal generator 430, is activated and the medical procedure is performed until a desired result occurs. For example, when the cardiac procedure is RF ablation, the RF generator is activated for a suitable period of time to create a lesion having a desired size. For example, lesion completion may be determined by monitoring changes in voltages observed in a transmyocardial bipolar electrogram, change in impedance value and/or loss of capture as is described with respect to FIGS. 7A-7C, for example.

Referring now to FIGS. 6A-6C, shown therein is an illustration of a process of forming a lesion with the bipolar catheter 100 having a spiral structure with the electrode 108. FIG. 6A shows that the active electrode 108 is rotatably inserted (e.g., screwed into) the cardiac tissue 602 so that the spiral structure encircles a target region 603 to be ablated. The spiral structure may be screwed into the cardiac tissue 602 until the electrode 110 of the cable body 102 makes contact with the surface 604 of the tissue 602 or is just above the surface (e.g., there may be a slight gap such as about 2 mm to about 4 mm). This enables the dispersive electrode 110 (i.e., the return electrode) to be positioned with respect to the tissue surface 204 so that proper impedance measurements can be made in at least one embodiment. Alternatively, increasing the size of the dispersive electrode 110 to increase the surface area of the of the dispersive electrode 110 may also increase the ability to obtain more accurate impedance measurements in cases where there is not full physical contact between the lower face (e.g., annular ring) of the dispersive electrode 110 and the upper portion of the tissue surface 604. FIG. 6A also shows the energy pathway between the fixated active electrode 108 and the dispersive electrode 110 when RF ablation is performed, and the Electric field lines extend from the active electrode 108 to the dispersive electrode 110 and deposit RF energy in the target cardiac region 603. FIG. 6B shows a lesion 604 that forms as the RF ablation is performed. The lesion has an outer boundary 606. FIG. 6C shows a top cross-sectional view of the active electrode 108 and the dispersive electrode 110 after RF ablation. In this case, the RF ablation is performed such that the lesion 604 extends past the geometric extent of the active electrode 108. The bipolar catheter may be moved to different cardiac locations where the RF ablation is repeated for all desired cardiac regions.

Accordingly, in at least one embodiment, the bipolar catheter 100 can be configured for use in bipolar ablation. In this use-case scenario, the active electrode 108 may be used as an anode and the dispersive electrode 110 may be used as a cathode. For ablation to occur properly, the impedance that is detected, by measuring bipolar or unipolar impedance, for example using the signal generator 430 or the measured signals may be analyzed by the bipolar catheter module 422, cannot reach a low extreme or a high extreme. For example, if the impedance reaches a high extreme such as greater than 240Ω for example, the ablation will fail as not enough power will be delivered to the targeted site. Conversely, when the impedance reaches a low extreme approaching approximately 50Ω, then the ablation will fail as this is the internal threshold for the RF generator.

In addition, in at least one embodiment, during the RF ablation, the impedance and temperature may be monitored to determine whether the operating conditions of the ablation catheter falls within safe limits (e.g., with respect to the measured temperature not exceeding an upper temperature threshold) and effective limits (e.g., with respect to the measured impedance staying within an acceptable operating range such as 50 to 250 Ohms, for example).

Alternatively other measurements may be made for monitoring the progress of RF ablation. For example, referring now to FIGS. 7A-7C, shown therein are illustrations of bipolar voltage reduction, signal capture and measured impedance, respectively, while performing RF ablation using one of the bipolar catheters described in accordance with the teachings herein. These figures provide an example of performing monitoring during RF ablation to determine when RF ablation procedure can be stopped. For example, FIG. 7A shows bipolar voltage that may be measured at the site of RF delivery using the bipolar catheter 200 that has the extra set of electrodes 204 and 206. The change in measured bipolar voltage may indicate the progress of RF ablation. For instance, the bipolar voltage measurement 700 in the example of FIG. 7A shows that the bipolar voltage dropped and there is 85% amplitude reduction post ablation by comparing measured signals 702 and 704 (which are preferably measured during pacing), suggesting that the formation of the lesion was complete. Therefore, monitoring of the bipolar voltage measurement over time at the site of RF ablation may be used to monitor lesion formation and determine that it is complete when a magnitude reduction for a second voltage that is measured post ablation compared to a first voltage that is measured pre ablation is greater than a magnitude reduction threshold.

Referring now to FIG. 7B, another technique for performing monitoring during RF ablation and lesion creation is to determine when there is loss of capture. This can be done while using a second set of electrodes, such as electrodes 204 and 206, to apply pacing and performing measurements 720 to determine when the QRS signal is no longer measured in at the target region. For example, during time period 722 the QRS signal can be measured but during time period 724 there is a loss of capture in the QRS signal. Accordingly, monitoring the voltage allows for determining the success of ablation. For example, if it took 1 my to capture a region of the myocardium before the ablation and post-ablation the same region of the myocardium cannot be captured at the highest output then this indicates that this region of the myocardium has been successfully fully ablated.

Referring now to FIG. 7C, another technique for performing monitoring during RF ablation and lesion creation is to measure the impedance at the target cardiac region and monitor how the measured impedance changes over time. For example, the plot 740 shows an impedance measurement curve 742 where the impedance decreases in magnitude over time as ablation is being performed and when the measured impedance value is smaller than an impedance threshold value it may indicate that the lesion is complete.

It should be noted that, prior to the teachings herein, there has not been an effective method to determine when lesion formation is complete. Conventionally, no one has considered monitoring a reduction in bipolar voltage in a transmural electrogram as a means for determining effective therapy delivery. Also no one has conventionally considered using the minimal amount of current/voltage needed to capture a signal from the myocardium as an indicator of myocardial viability.

Alternatively, referring again to FIG. 5, at 506, when the cardiac procedure is transmural mapping or pace mapping, a suitable stimulus is provided, and a suitable amount of cardiac data is measured to perform the mapping. The bipolar catheter may be moved to different cardiac locations where the mapping or pacing is repeated until cardiac data is obtained for all desired cardiac regions. During mapping or pacing, measured temperature and/or measured impedance may be monitored to make sure that the pacing and/or mapping is being done safely, as described previously. It should be noted that the bipolar catheters described herein may allow for intramural pacing, which was not possible with previous catheters.

Alternatively, at 506 of method 500, in at least one embodiment, one of the bipolar catheters described herein can be configured for use in transmural mapping. The electrode at the tip of the spiral structure together with the other electrode, which is generally in the shape of a ring, allows for functions as a mapping tool, including as a transmural mapping tool. In this case, the active electrode terminal may be connected to the signal generator 430 that acts as a stimulator.

In at least one embodiment, one of the bipolar catheters described herein can be configured for use in pace mapping. The electrode at the tip of the spiral structure together with the other electrode, which is generally in the shape of a ring, allows for functions as a pace mapping tool, including as a transmural pace mapping catheter. In this case, the active electrode terminal may be connected to the signal generator 430 that acts as a stimulator.

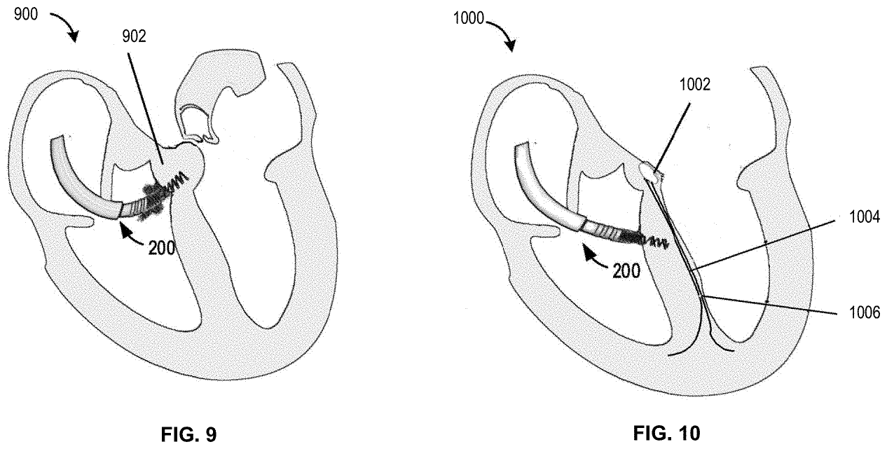

Referring now to FIGS. 8A and 8B, in at least one alternative embodiment, a bipolar catheter 800 may have a different arrangement for the electrodes. In this case, the electrodes are smaller in nature and disposed to provide more than two bipoles that are tightly spaced. For example, the catheter 800 has a helical structure 802 with eight small electrodes that are placed within the pitches of the helical structure 802 and that are wired to provide four tightly spaced bipoles 804, 806, 808, and 810 which allows for high density intramural bipolar mapping. Such a multi-bipole electrode structure allows for bipolar and unipolar mapping to assess activation and repolarization intramurally. In making the structure 802 with the eight electrodes (shown as plus or minus signs) the helical structure is mostly insulated but there are eight small regions that are not insulated and are connected to eight separate wires to provide the four bipoles 804 to 810. In alternatives of this embodiment, there may be a different number of bipoles but there are at least two bipoles. In an example embodiment, the electrodes may be 1 mm in size with a 2 mm centre to centre spacing for the bipoles. In alternative embodiments, the bipolar catheter 800 may include a conduit for providing medical agents near the helical structure 802 as in the embodiments and alternatives discussed for bipolar catheter 200 or 300. In some cases, the bipolar catheter 800 may be used with an electroporation energy source.

It should be noted that there may be alternative applications for the bipolar catheter embodiments described herein which may be performed at 506 of method 500.

Referring now to FIG. 9, shown therein is an illustration showing an example use of the bipolar catheter 200 for cardiac debulking. For example, when a patient's heart has hypertrophic cardiomyopathy conventionally what is done the thick areas of the myocardium are shaved off. This patient would have to go to the Operating Room (OR) where they must undergo surgery and have their chest opened on to gain access to the heart to perform the procedure. However, with the bipolar catheters described herein ablation can be performed to ablate and shrink the tissue. For example, in FIG. 9 the bipolar catheter 200 may be inserted via the vascular to a desired target region of a heart 900 where the cardiac muscle has been thickened 902 and then RF ablation can be used to shrink the muscle thickness. During the RF ablation fluid may be dispersed.

Referring now to FIG. 10, shown therein is an illustration showing an example use of the bipolar catheter 200 for determining a suitable location for electrode placement for a pacemaker. For example, when placing pacemaker 1002 having electric leads 1004 and 1006 into heart 1000, it is helpful to use a bipolar catheter, such as bipolar catheter 200, as a scout catheter and use pacing to determine the regions of the heart where the leads 1004 and 1006 can be inserted.

As another example, at least one example embodiment of the bipolar catheters described herein can be used as a diagnostic catheter in the septum to determine if a left bundle branch block (LBBB) or a right bundle branch block (RBBB) may be narrowed before attempting physiological pacing. Conventionally this might have been done using 12 lead surface ECG.

In example applications, when at least one embodiment of the bipolar catheters described herein is used to perform RF ablation, certain cardiac tissues can be ablated. For instance, at least one embodiment of the bipolar catheter described herein may be used for: (1) the ablation of papillary muscle which causes arrhythmias, (2) the ablation of tissue for hemodynamic reasons for treatment of hypertrophic cardiomyopathy including obstructive septum from the venous approach without having to send the patient for open heart surgery or alcohol ablation, (3) shrinking cardiac tumors with ablation therapies.

In another example application, when at least one embodiment of the bipolar catheters described herein is used to perform RF ablation, it may be used for treating certain congenital heart conditions by creating access to in accessible regions of the heart. For example, in congenital hearts there are baffles that may be penetrated by screwing into the wall of the baffle and performing ablation to gain access to previously inaccessible regions.

In another example application, at least one embodiment of the bipolar catheters described herein can be used for treating other types of tissues rather than just cardiac tissues. For example, at least one embodiment of the bipolar catheter described herein can be used for treating liver and renal tumors, the lungs, the bladder and the brain with a trans-vascular approach.

### Experimental Protocol for Determining Effective Electrode Configurations

To gain a better understanding on how lesions are formed using the bipolar catheters described herein, various embodiments having different spatial positioning were tested while RF generator settings were varied. In general, a Stockert RF generator (Biosense Webster) was used for the experiments. The advantage of using this unit was that it was able to disable certain thresholds that may potentially interrupt the ablation process. However, this does not disable hardwired thresholds for hardware and/or patient safety. Table 1 indicates parameter settings that were disabled before proceeding with the ablations.

TABLE 1
Parameters that were disabled for bipolar ablation experiments

| Parameter/Settings | Status |
|---|---|
| Impedance Min | OFF |
| Impedance Max | OFF |
| Impedance Delta | OFF |
| Voltage Limiter | OFF |
| Current Limiter | OFF |

### Experiment #1—Results

By varying different combinations of screw electrodes, return electrodes, and power levels different lesions were formed. For example, electrodes placed on different spiral structures and materials were used for the experiments where the helical diameter/pitch varied from 2-2.5 mm, the wire diameter varied from 0.3-0.5 and the material that was used included platinum coated steel or a Nickel-alloy. Unless specified within the respective experimental results section herein, all ablations were conducted for about 30 seconds. Lesion depth and width recordings were taken to evaluate the effectiveness of the tested electrodes. Each experiment that was documented properly is tabulated and briefly described in their respective section.

Table 2 indicates the results of an experiment performed on a pig Langendorff experiment. The bipolar catheter configuration included a 5 mm screw silver electrode and a return silver electrode located at the epicardial surface of the heart. The screw electrode was used as the active electrode and the epicardial electrode was used as the dispersive electrode. The unipolar configuration used a large silver electrode located away from the ablation site as a return electrode. Changes of voltage were measured between pre ablation and post ablation. Overall changes of the peak-to-peak voltages (e.g., segment decrement) and averaged values of the peak-to-peak voltages were measured for each individual beats. Each segment included 8 to 10 beats. The % values are expressed from 100% pre-ablation levels.

TABLE 2
Peak-to-peak voltage changes for different
power levels and ablation modes

| | Vpp (Segment) | Vpp (Averaging) | Impedance |
|---|---|---|---|
| Power (W) | Decrement | Change | Decrement (Ω) |
| 8 W Unipolar | −0.7387 mV | −0.9143 mV | Pre: 121 |
| | −32.71% | −41.95% | Post: 174 |
| 8 W Bipolar | −4.6716 mV | −4.8607 mV | Pre: 135 |
| | −61.75% | −70.68% | Post: 114 |
| 16 W Unipolar | +0.4516 mV | −0.15 mV | Pre: 125 |
| | +27.93% | −10.37% | Post: — |
| 16 W Bipolar | −4.2372 mV | −4.5116 mV | Pre: 170 |
| | −61.13% | −67.53% | Post: — |

### Experiment #2—Results

In this set of experiments, different material was used, and the positioning of electrodes were varied. Although lesions were formed at least once for each configuration, the RF generator was not delivering enough power and was throttling. In instances when the impedance exceeded 1,000Ω for a short duration, the RF generator produced an error message indicating an overflow of current. This caused the RF generator to shut off and require a system reset.

For the results shown in Table 3, the electrode setup #1 consisted of the active electrode being an insulated metal screw that penetrated into the tissue, and the dispersive electrode being an exposed silver screw that was located on the tissue surface. In this pilot study, the optimal power and electrode arrangement was investigated while leaving the RF duration at 30 seconds. Table 3 shows initial results with lower power (10 W). While the lesion from one application of RF energy was measured and located, it was hard to find a second lesion, so a higher power level was used as shown in Table 4.

TABLE 3
Summary of Bipolar ablation results for electrode setup #1

| | | |
|---|---|---|
| Power | Measurement (mm) | Impedance (Ω) |

| Lesion | (W) | Length | Depth | Before | After |
|---|---|---|---|---|---|
| 1 | 10 | — | — | 220 | 150 |
| 2 | 10 | 2.56 | 1.61 | 230 | 115 |

For the results shown in Table 4, the electrode setup #2 consisted of the active electrode being an insulated metal screw that penetrated into the tissue, and the dispersive electrode being an exposed silver screw that also penetrated the tissue. For these results, the power level was established at 30 W, and the dispersive electrode was inserted into the tissue to increase lesion size control. During the experiment, power delivery was also throttled below 30 W.

TABLE 4
Summary of Bipolar ablation results for electrode setup #2

| | | |
|---|---|---|
| Power | Measurement (mm) | Impedance (Ω) |

| Lesion | (W) | Length | Depth | Before | After |
|---|---|---|---|---|---|
| 1 | 30 | 2.24 | 0.88 | 720 | 580 |
| 2 | 30 | — | — | 200 | — |
| 3 | 30 | 2.24 | 1.17 | 170 | — |

For the results shown in Table 5, the electrode setup #3 consisted of the active electrode being an insulated needle that penetrated into the tissue, and the dispersive electrode being an exposed silver screw that also penetrated the tissue. The protocol for this experiment was the same as the previous experiment, except a needle was used as the active electrode. It was thought that using a needle as the active electrode would create a different current flow. During this experiment, power throttling was observed which had an impact on lesion sizes, although larger lesion depths were observed.

TABLE 5
Summary of Bipolar ablation results for electrode setup #3

| | | |
|---|---|---|
| Power | Measurement (mm) | Impedance (Ω) |

| Lesion | (W) | Length | Depth | Before | After |
|---|---|---|---|---|---|
| 1 | 30 | — | — | 260 | — |
| 2 | 30 | 2.19 | 0.91 | 190 | 120 |
| 3 | 30 | 2.29 | 4.10 | 230 | 210 |
| 4 | 30 | 5.56 | 4.35 | 225 | 123 |

### Experiment #3—Results

In this set of experiments, different needle lengths were used for the dispersive electrode. The electrode setup #4 consisted of the active electrode being a screw electrode that was placed in the tissue, and the dispersive electrode being a needle that pierced the tissue through the center of the screw in attempt to minimize the dispersion of energy. Although 30 W was used, the power was throttling and limited the actual amount of power to about 20 W. During RF ablation for the creation of lesion 4, the lesion was not visible and thus, no measurements were taken. The only transmural lesion was from Lesion 7. Table 6 shows the results for different coil electrode exposures and Table 7 shows the results for different needle exposure.

TABLE 6
Bipolar ablation summary of electrode setup #4

| | Electrode | | | | | |
|---|---|---|---|---|---|---|
| | Exposure | Power | Measurement (mm) | | Impedance (Ω) | |
| Lesion | (mm) | (W) | Length | Depth | Before | After |
| 1 | 7 | 30 | 4.59 | 3.44 | 60 | 30 |
| 2 | 5 | 30 | 1.30 | 12.18 | 330 | 120 |
| 3 | 5 | 30 | 4.17 | 12.36 | 160 | 110 |
| 4 | 5 | 30 | — | — | 160 | 100 |
| 5 | 10 | 30 | 3.24 | 9.66 | 107 | 50 |
| 6 | 10 | 30 | 3.52 | 8.89 | 200 | 100 |
| 7 | 7 | 30 | 3.90 | 14.90 | 80 | 70 |

TABLE 7
Bipolar ablation summary of electrode setup #4

| | Needle | | | | | |
|---|---|---|---|---|---|---|
| | Exposure | Power | Measurement (mm) | | Impedance (Ω) | |
| Lesion | (mm) | (W) | Length | Depth | Before | After |
| 1 | 5 | 30 | 2.18 | 5.45 | 104 | 52 |
| 2 | 5 | 30 | 1.91 | 16.86 | 300 | 110 |
| 3 | 10 | 30 | 3.44 | 12.49 | 140 | 40 |

For the results shown in Table 8, the electrode setup #5 consisted of the active electrode being an insulated needle that penetrated into the tissue, and the dispersive electrode being an exposed silver screw that also penetrated the tissue.

TABLE 8
Bipolar ablation summary of electrode setup #5

| | Needle | | | | | |
|---|---|---|---|---|---|---|
| | Exposure | Power | Measurement (mm) | | Impedance (Ω) | |
| Lesion | (mm) | (W) | Length | Depth | Before | After |
| 1 | 5 | 30 | 5.90 | 3.89 | 300 | 40 |
| 2 | 5 | 30 | 2.94 | 3.67 | 130 | 30 |
| 3 | 10 | 30 | 2.31 | 7.11 | 600 | — |

### Experiment #4—Results

In this experiment, the duration for performing RF ablation was only varied for Lesion 1 from 30 seconds to 60 seconds but was terminated early. In addition, in Lesion 1, the active and dispersive electrodes were placed adjacent to each other. For the remaining lesions, the RF ablations were carried out for a 30 second duration and electrode setup #6 consisted of the active electrode being a titanium screw electrode that was inserted into the tissue within the dispersive electrode which was a ring electrode that was placed on the tissue. All ablations were performed epicardially.

Impedance measurements were not recorded as the objective was to observe the effectiveness of the electrode combination. Both active and dispersive electrodes were made using titanium wire, which was heat treated as described previously. This experiment was done to find a conductive material that is less ductile. The results are shown in Table 9. This experiment showed that the electrode arrangement provided the desired functionality, with ablation sizes comparable to the data shown in Tables 6 and 7.

TABLE 9
Bipolar ablation summary of electrode setup #6

| | | |
|---|---|---|
| Power | Measurement (mm) | Impedance (Ω) |

| Lesion | (W) | Length | Depth | Before | After |
|---|---|---|---|---|---|
| 1 | 30 | 11.18 | 5.81 | — | — |
| 2 | 30 | 14.64 | 8.11 | — | — |
| 3 | 15 | 11.53 | 5.38 | — | — |
| 4 | 10 | 8.62 | 7.50 | — | — |

### Conclusion

The feasibility of a single catheter to perform bipolar ablation, with an electrode on a spiral structure to provide a deep and controllable lesion has been demonstrated with encouraging results based on the lesions formed. The experimental results show that electrode sizing influences the overall size of the lesion. Based on our current finding, in order to maintain the screw length to be approximately 10 mm, the return or dispersive electrode was successful in forming lesions when it was a 2-3 mm exposed wire. While some impedance settings for the RF generator were disabled to increase the probability of the ablation procedure to occur, the lesions that were formed had considerable depth and localized length. However, sufficient tissue thickness is required for the formation of proper lesions.

The experimental results also showed that lesions were still forming when there was indirect contact between the dispersive electrode and the tissue surface, which means that bipolar catheter embodiments can be used where the dispersive electrode is positioned more proximally on the catheter body (e.g., shaft of the catheter) but in this case the surface area of the dispersive electrode may be increased.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. A bipolar catheter comprising:
a catheter body having a distal end portion and a proximal end portion;
a first electrode at the distal end portion, the first electrode is provided on a spiral structure for rotational insertion into a physiological target region;
a second electrode at the proximal end portion and spaced apart from the first electrode; and
first and second electrode terminals spaced apart from one another at the proximal end portion and electrically coupled to the first and second electrodes respectively,
wherein the first and second electrodes are configured to function as active and dispersive electrodes respectively, or the first and second electrodes are configured to function as dispersive and actives electrodes, respectively.

2. The bipolar catheter of claim 1, wherein the first electrode is part of a coil electrode and the active electrode has an insulated portion and a conductive surface portion.

3. The bipolar catheter of claim 1, wherein the dispersive electrode is a ring electrode that is disposed about a circumference of the distal end portion.

4. The bipolar catheter claim 1, wherein the bipolar catheter comprises a conduit that has a first end that is connectable to a source of one or more medical agents and a second end at the distal end of the catheter and during use the one or more medical agents flow through the conduit and are delivered at the distal end portion of the bipolar catheter.

5. The bipolar catheter of claim 1, wherein the bipolar catheter comprises a conduit that has a first end that is connectable to a fluid source and a second end at the distal end of the catheter and during use fluid from the fluid source is flowed through the conduit and delivered at the distal end portion of the bipolar catheter for irrigation and/or cooling.

6. The bipolar catheter of claim 1, wherein the bipolar catheter comprises first and second conduits with the first conduit having a first end that is connectable to a source of one or more medical agents and a second end that is disposed at the distal end of the bipolar catheter and the second conduit having a first end that is connectable to a fluid source and a second end that is disposed at the distal end of the bipolar catheter and during use the one or more medical agents flow through the first conduit and are delivered at the distal end portion of the bipolar catheter and/or fluid from the fluid source is flowed through the second conduit and delivered at the distal end portion of the bipolar catheter for irrigation.

7. The bipolar catheter of claim 4, wherein the first electrode has pores and the first electrode is connected to the conduit for receiving the one or more medical agents during use.

8. The bipolar catheter of claim 5, wherein the second electrode has a porous structure and the second electrode is connected to the conduit for receiving the fluid for irrigation and/or cooling during use.

9. The bipolar catheter of claim 1, wherein the catheter further comprises an adjustment mechanism that is mechanically coupled to the first electrode and configured to controllably adjust a variable depth of penetration of a tip of the first electrode away from or closer to an end face of the catheter body at the distal end portion.

10. The bipolar catheter of claim 1, wherein the fluoroscopic markers are attached to the spiral structure to allow for visual confirmation of the depth of the tip of the spiral structure during use via fluoroscopic imaging.

11. The bipolar catheter of claim 1, wherein the bipolar catheter comprises a second pair of electrodes configured for measuring voltages thereat and/or the first electrode is on a portion of the spiral structure and the spiral structure comprises a plurality of additional electrodes for providing at least two bipoles for performing high density intramural bipolar mapping or unipolar mapping.

12. The bipolar catheter of claim 1, wherein the physiological target region comprises cardiac tissue and the bipolar catheter is configured for use in bipolar ablation, transmural mapping, pace mapping, cardiac debulking and/or monitoring lesion formation.

13. A method of performing a procedure using a bipolar catheter, wherein the method comprises:
coupling the bipolar catheter to a signal generator, the bipolar catheter being defined according to claim 1;
inserting the bipolar catheter at a physiological target region; and
performing the procedure.

14. The method of claim 13, wherein the bipolar catheter is inserted at an initial tissue region until the second electrode contacts a surface of the physiological target region.

15. The method of claim 13, wherein the physiological target region comprises cardiac tissue and the procedure comprises performing bipolar ablation, transmural mapping, pace mapping, cardiac debulking and/or monitoring lesion formation.

16. The method of claim 13, wherein the bipolar catheter further comprises a temperature sensor at a distal end portion of the catheter body and the procedure comprises performing bipolar ablation or tissue debulking and the method comprises measuring a temperature at the target tissue region and stopping the bipolar ablation when the measured temperature is higher than a temperature threshold.

17. The method of claim 13, wherein the procedure comprises performing bipolar ablation or tissue debulking and the method comprises measuring an impedance at a distal end portion of the bipolar catheter and performing the bipolar ablation or tissue debulking while the measured impedance is within an effective operating range or comparing the measured impedance to an impedance threshold to determine when the ablation or debulking has been done for a sufficient amount of time.

18. The method of claim 13, wherein the procedure comprises performing bipolar ablation or tissue debulking and the method comprises measuring a first voltage before the procedure and a second voltage after the procedure, determining a magnitude reduction for the second voltage compared to the first voltage and determining that the medical procedure was successful when the magnitude reduction is greater than a magnitude reduction threshold.

19. The method of claim 13, wherein the procedure comprises performing bipolar ablation or tissue debulking and the method comprises measuring voltage during the procedure and determining that the procedure is complete when there is a loss in signal capture.

20. The method of claim 13, wherein the procedure comprises delivering fluid to the physiological target region to provide cooling and/or reduce formation of char during RF ablation.

21. The method of claim 13, wherein the procedure comprises delivering one or more medical agents to the physiological target region, the one or more medical agents comprising one or more therapeutic agents, one or more diagnostic agents and/or one or more marker agents.

22. The method of claim 21, wherein the one or more medical agents comprise pharmaceuticals, antiarrhythmics, MRNA and DNA to reprogram cells in the physiological target region, stem cells, viral vectors to reprogram existing cells in the physiological target region, biological pacemakers, radiology markers for later targeting therapy with radiation or a gamma knife and/or biological reporters.
